(19) United States
(12) Patent Application Publication
(10) Pub. No.: US 2018/0113185 A1
(43) Pub. Date: Apr. 26, 2018

(54) SYSTEM AND METHOD FOR IMAGING NANODIAMONDS AS DYNAMIC NUCLEAR POLARIZATION AGENT

(71) Applicant: The General Hospital Corporation, Boston, MA (US)

(72) Inventors: Matthew S. Rosen, Somerville, MA (US); Mathieu Sarracanie, Somerville, MA (US); Huiliang Zhang, Cambridge, MA (US); David Riley, University of Sydney (AU); Ronald L. Walsworth, Newton, MA (US); David Waddington, Cambridge, MA (US)

(21) Appl. No.: 15/562,551

(22) PCT Filed: Apr. 1, 2016
(86) PCT No.: PCT/US16/25543

§ 371 Date: Sep. 28, 2017

Related U.S. Application Data
(60) Provisional application No. 62/141,507, filed on Apr. 1, 2015.

(51) Int. Cl. G01R 33/56; G01R 33/28; G01R 33/44; G01R 33/561; G01R 33/62; A61B 5/055; A61K 49/18

(52) U.S. Cl. CPC G01R 33/5601; G01R 33/282; G01R 33/445; A61K 49/1818; G01R 33/62; A61B 5/055; G01R 33/5614

(57) ABSTRACT

A system and method performing a medical imaging process includes arranging a subject to receive solution comprising nanodiamonds, performing an MRI imaging process to acquire data from the subject, and reconstructing the data to generate a report indicating a spatial distribution of nanodiamonds in the subject.

### CROSS-REFERENCE TO RELATED APPLICATIONS

This application is based on, claims priority to, and incorporates herein by reference, U.S. Provisional Application Ser. No. 62/141,507, filed Apr. 1, 2015, and entitled “NANODIAMONDS AS DYNAMIC NUCLEAR POLARIZATION AGENT FOR MRI.”

### STATEMENT REGARDING FEDERALLY SPONSORED RESEARCH

This disclosure was made with government support under W81XWH 11-2-076 awarded by the Department of Defense. The government has certain rights in the disclosure.

### FIELD

The present disclosure relates to systems and methods for magnetic resonance imaging (MRI). More particularly, the present disclosure provides systems and methods for imaging nanodiamonds using MRI and probes.

### BACKGROUND

When a substance such as human tissue is subjected to a uniform magnetic field (polarizing field B₀), the individual magnetic moments of the excited nuclei in the tissue attempt to align with this polarizing field, but precess about it in random order at their characteristic Larmor frequency. If the substance, or tissue, is subjected to a magnetic field (excitation field B₁) which is in the x-y plane and which is near the Larmor frequency, the net aligned moment, M_{z}, may be rotated, or “tipped”, into the x-y plane to produce a net transverse magnetic moment Mₜ. A signal is emitted by the excited nuclei or “spins”, after the excitation signal B₁ is terminated, and this signal may be received and processed to form an image.

When utilizing these “MR” signals to produce images, magnetic field gradients (Gₓ, G_{y}, and G_{z}) are employed. Typically, the region to be imaged is scanned by a sequence of measurement cycles in which these gradients vary according to the particular localization method being used. The resulting set of received MR signals are digitized and processed to reconstruct the image using one of many well known reconstruction techniques.

Acute reperfusion therapies have changed ischemic stroke care, but treatments are limited because of a short therapeutic window owing to the risk of reperfusion injury and hemorrhage. Detection of early and mild blood-brain barrier (BBB) disruption is an unmet need in acute stroke diagnosis. Although contrast from relaxation-based MRI contrast agents such as Gd-DTPA is correlated with hemorrhagic transformation of an infarct, it is not sensitive enough to probe more mild BBB disruption.

Thus, there is a need to provide additional systems or methods facilitate in vivo analysis of pathologies, such as stroke, reperfusion injury, hemorrhage, BBB disruption, and other vascular conditions.

### SUMMARY

The present disclosure provides systems and methods that overcome the aforementioned drawbacks by providing a system and method for MRI in conjunction with injected nanodiamonds. Nontoxic nanodiamonds (NDs) have proven useful as a vector for therapeutic drug delivery to cancers, and as optical bioprobes of subcellular processes. The systems and methods provided herein provide a means of noninvasively imaging NDs in vivo using MRI.

In accordance with one aspect of the disclosure, a MRI system is disclosed that is configured to perform an imaging process of a subject having received nanodiamonds. The MRI system includes a magnet system configured to generate a static magnetic field about at least a region of interest (ROI) of a subject arranged in the MRI system. The MRI system includes at least one gradient coil configured to establish at least one magnetic gradient field with respect to the static magnetic field. The system also includes a radio frequency (RF) system configured to deliver excitation pulses to the subject. The computer system is programmed to: control the at least one gradient coil and the RF system to perform a MRI pulse sequence; acquire data corresponding to signals from the subject having received solution comprising nanodiamonds; and reconstruct, from the data, at least one anatomical image of the subject and spatially distributed nanodiamonds within the subject relative to the anatomical image.

In accordance with another aspect of the disclosure, a method is provided for performing a medical imaging process. The method includes arranging a subject to receive solution comprising nanodiamonds and performing a magnetic resonance imaging (MRI) process to acquire a first data from the subject. The method also includes performing an Overhauser-enhanced magnetic resonance imaging (OMRI) process to acquire a second data from the subject and reconstructing the first and second data to generate a report indicating a spatial distribution of the nanodiamonds in the subject.

In accordance with another aspect of the disclosure, a magnetic resonance imaging (MRI) system is provided that includes a magnet system configured to generate a static magnetic field about at least a region of interest (ROI) of a subject arranged in the MRI system and at least one gradient coil configured to establish at least one magnetic gradient field with respect to the static magnetic field. The MRI system also includes a radio frequency (RF) system configured to deliver excitation pulses to the subject having received solution comprising nanodiamonds, wherein the excitation pulses comprises at least one embedded electron paramagnetic resonance (EPR) pulse and a controller configured to manipulate dynamic nuclear polarization (DNP) contrast caused by the nanodiamonds by turning on or turning off the at least one embedded electron paramagnetic resonance (EPR) pulse.

The foregoing and other advantages of the disclosure will appear from the following description.

### BRIEF DESCRIPTION OF THE DRAWINGS

FIG. 1 is a block diagram of an MRI system configured in accordance with the present disclosure.

FIG. 2 is a block diagram of an RF system of an MRI system configured in accordance with the present disclosure.

FIG. 3 is a picture of a low-field MRI (IfMRI) system in accordance with the present disclosure.

FIG. 4a is a picture of a probe for OMRI in accordance with the present disclosure.

FIG. 4b illustrates the structure of a probe for OMRI in accordance with the present disclosure.

FIG. 4c illustrates the simulation result of the electric field of the probe in FIG. 4b in accordance with the present disclosure.

FIG. 4d illustrates the simulation result of B₁ₑ field of the probe in FIG. 4b in accordance with the present disclosure.

FIG. 5a is a pulse sequence diagram for a pulse sequence in accordance with the present disclosure.

FIG. 5b illustrate an example of steps of a method in accordance with the present disclosure.

FIG. 5c illustrate additional acts that may be implemented in accordance with the present disclosure.

FIG. 6 shows images acquired from a rat using a system in accordance with the present disclosure.

FIG. 7a shows nanodiamonds dispersed in water is a source of paramagnetic impurities.

FIG. 7b shows RF drives Rabi oscillations, transferring polarization from electrons to nuclei.

FIG. 8a shows MRI images of a ND phantom acquired using a system in accordance with the present disclosure.

FIG. 8b shows DNP MRI images of a ND phantom acquired using a system in accordance with the present disclosure.

FIG. 8c shows the difference image of images in FIG. 8a and FIG. 8b.

FIG. 8d shows schematic of phantom vials.

FIG. 9 shows ¹H NMR spectra at 6.5 mT from a 100 mg/mL solution of MSY18 NDs.

FIG. 10 shows the ¹H enhancement at a constant ESR frequency.

### DETAILED DESCRIPTION

Magnetic resonance imaging (MRI) is a powerful non-invasive technology that provides a unique window to the structure and the function of the body, with high resolution, speed, and biological contrast. To extend the diagnostic potential of MRI to NDs, much effort were put into using intrinsic paramagnetic impurities in the ND to hyperpolarize 13C in the ND core. The present disclosure advantageously recognizes that naturally occurring paramagnetic impurities in ND may also couple to ¹H nuclei in water. To this end, the systems and methods of the present disclosure can exploit this coupling to non-invasively image concentrations of NDs in aqueous environment. Overhauser-enhanced MRI (OMRI) can be used in conjunction with an ultra-low field MRI scanner to image synthetic nanodiamonds (NDs) in water at room temperature to obtain the first reported OMRI images of ND.

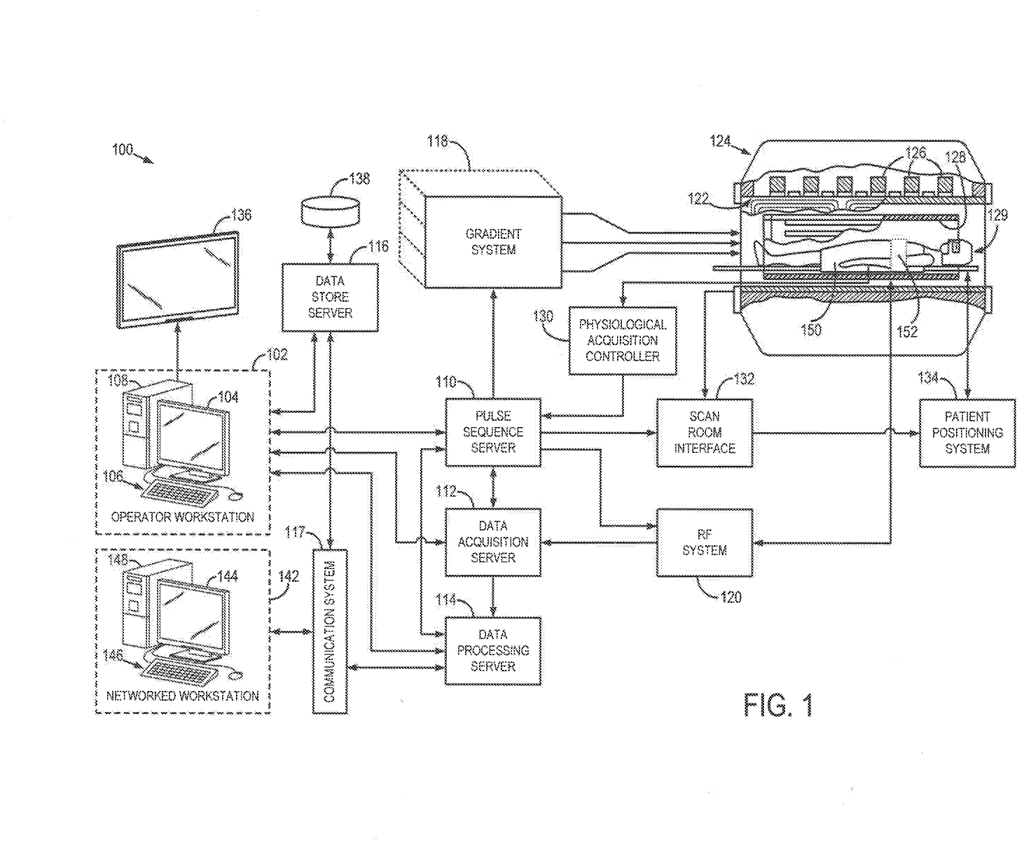

Referring particularly now to FIG. 1, an example of a magnetic resonance imaging (MRI) system 100 is illustrated. The MRI system 100 includes an operator workstation 102, which will typically include a display 104, one or more input devices 106, such as a keyboard and mouse, and a processor 108. The processor 108 may include a commercially available programmable machine running a commercially available operating system. The operator workstation 102 provides the operator interface that enables scan prescriptions to be entered into the MRI system 100. In general, the operator workstation 102 may be coupled to four servers: a pulse sequence server 110; a data acquisition server 112; a data processing server 114; and a data store server 116. The operator workstation 102 and each server 110, 112, 114, and 116 are connected to communicate with each other. For example, the servers 110, 112, 114, and 116 may be connected via a communication system 117, which may include any suitable network connection, whether wired, wireless, or a combination of both. As an example, the communication system 117 may include both proprietary or dedicated networks, as well as open networks, such as the internet.

The pulse sequence server 110 functions in response to instructions downloaded from the operator workstation 102 to operate a gradient system 118 and a radiofrequency (“RF”) system 120. Gradient waveforms necessary to perform the prescribed scan are produced and applied to the gradient system 118, which excites gradient coils in an assembly 122 to produce the magnetic field gradients Gₓ, G_{y}, and G_{z} used for position encoding magnetic resonance signals. The gradient coil assembly 122 forms part of a magnet assembly 124 that includes a polarizing magnet 126 and a whole-body RF coil 128 and/or local coil, such as a head coil 129.

The MRI system 100 may specify a region of interest (ROI) 152 in the subject 150 by manipulating the gradient system 118 and the RF system 120. The MRI system 100 may apply additional transmitting or receiving coils to image an ROI 152 in the subject 150.

RF waveforms are applied by the RF system 120 to the RF coil 128, or a separate local coil, such as the head coil 129, in order to perform the prescribed magnetic resonance pulse sequence. Responsive magnetic resonance signals detected by the RF coil 128, or a separate local coil, such as the head coil 129, are received by the RF system 120, where they are amplified, demodulated, filtered, and digitized under direction of commands produced by the pulse sequence server 110. The RF system 120 includes an RF transmitter for producing a wide variety of RF pulses used in MRI pulse sequences. The RF transmitter is responsive to the scan prescription and direction from the pulse sequence server 110 to produce RF pulses of the desired frequency, phase, and pulse amplitude waveform. The generated RF pulses may be applied to the whole-body RF coil 128 or to one or more local coils or coil arrays, such as the head coil 129.

The RF system 120 also includes one or more RF receiver channels. Each RF receiver channel includes an RF preamplifier that amplifies the magnetic resonance signal received by the coil 128/129 to which it is connected, and a detector that detects and digitizes the I and Q quadrature components of the received magnetic resonance signal. The magnitude of the received magnetic resonance signal may, therefore, be determined at any sampled point by the square root of the sum of the squares of the I and Q components:

M=\sqrt{I²+Q²} (1);

and the phase of the received magnetic resonance signal may also be determined according to the following relationship:

$$ϕ = {tan}^{- 1} ( \frac{Q}{I} ) . \qquad ( 2 )$$

The pulse sequence server 110 also optionally receives patient data from a physiological acquisition controller 130. By way of example, the physiological acquisition controller 130 may receive signals from a number of different sensors connected to the patient, such as electrocardiograph (“ECG”) signals from electrodes, or respiratory signals from a respiratory bellows or other respiratory monitoring device. Such signals are typically used by the pulse sequence server 110 to synchronize, or “gate,” the performance of the scan with the subject's heart beat or respiration.

The pulse sequence server 110 also connects to a scan room interface circuit 132 that receives signals from various sensors associated with the condition of the patient and the magnet system. It is also through the scan room interface circuit 132 that a patient positioning system 134 receives commands to move the patient to desired positions during the scan.

The digitized magnetic resonance signal samples produced by the RF system 120 are received by the data acquisition server 112. The data acquisition server 112 operates in response to instructions downloaded from the operator workstation 102 to receive the real-time magnetic resonance data and provide buffer storage, such that no data is lost by data overrun. In some scans, the data acquisition server 112 does little more than pass the acquired magnetic resonance data to the data processor server 114. However, in scans that require information derived from acquired magnetic resonance data to control the further performance of the scan, the data acquisition server 112 is programmed to produce such information and convey it to the pulse sequence server 110. For example, during prescans, magnetic resonance data is acquired and used to calibrate the pulse sequence performed by the pulse sequence server 110. As another example, navigator signals may be acquired and used to adjust the operating parameters of the RF system 120 or the gradient system 118, or to control the view order in which k-space is sampled. In still another example, the data acquisition server 112 may also be employed to process magnetic resonance signals used to detect the arrival of a contrast agent in a magnetic resonance angiography (MRA) scan. By way of example, the data acquisition server 112 acquires magnetic resonance data and processes it in real-time to produce information that is used to control the scan.

The data processing server 114 receives magnetic resonance data from the data acquisition server 112 and processes it in accordance with instructions downloaded from the operator workstation 102. Such processing may, for example, include one or more of the following: reconstructing two-dimensional or three-dimensional images by performing a Fourier transformation of raw k-space data; performing other image reconstruction algorithms, such as iterative or backprojection reconstruction algorithms; applying filters to raw k-space data or to reconstructed images; generating functional magnetic resonance images; calculating motion or flow images; and so on.

Images reconstructed by the data processing server 114 are conveyed back to the operator workstation 102 where they are stored. Real-time images are stored in a data base memory cache (not shown in FIG. 1), from which they may be output to operator display 112 or a display 136 that is located near the magnet assembly 124 for use by attending physicians. Batch mode images or selected real time images are stored in a host database on disc storage 138. When such images have been reconstructed and transferred to storage, the data processing server 114 notifies the data store server 116 on the operator workstation 102. The operator workstation 102 may be used by an operator to archive the images, produce films, or send the images via a network to other facilities.

The MRI system 100 may also include one or more networked workstations 142. By way of example, a networked workstation 142 may include a display 144; one or more input devices 146, such as a keyboard and mouse; and a processor 148. The networked workstation 142 may be located within the same facility as the operator workstation 102, or in a different facility, such as a different healthcare institution or clinic.

The networked workstation 142, whether within the same facility or in a different facility as the operator workstation 102, may gain remote access to the data processing server 114 or data store server 116 via the communication system 117. Accordingly, multiple networked workstations 142 may have access to the data processing server 114 and the data store server 116. In this manner, magnetic resonance data, reconstructed images, or other data may exchanged between the data processing server 114 or the data store server 116 and the networked workstations 142, such that the data or images may be remotely processed by a networked workstation 142. This data may be exchanged in any suitable format, such as in accordance with the transmission control protocol (TCP), the internet protocol (IP), or other known or suitable protocols.

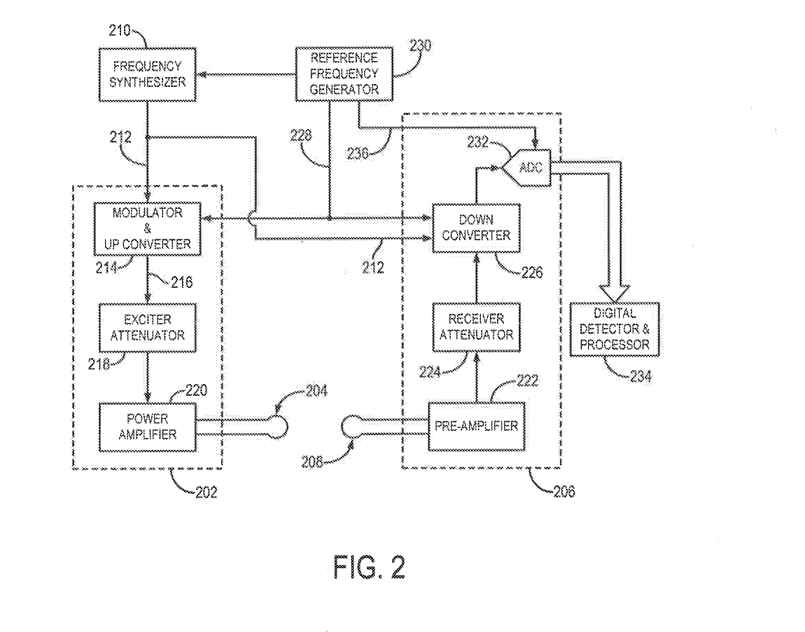

With reference to FIG. 2, the RF system 120 of FIG. 1 will be further described. The RF system 120 includes a transmission channel 202 that produces a prescribed RF excitation field. The base, or carrier, frequency of this RF excitation field is produced under control of a frequency synthesizer 210 that receives a set of digital signals from the pulse sequence server 110. These digital signals indicate the frequency and phase of the RF carrier signal produced at an Output 212. The RF carrier is applied to a modulator and up converter 214 where its amplitude is modulated in response to a signal, R(t), also received from the pulse sequence server 110. The signal, R(t), defines the envelope of the RF excitation pulse to be produced and is produced by sequentially reading out a series of stored digital values. These stored digital values may be changed to enable any desired RF pulse envelope to be produced.

The magnitude of the RF excitation pulse produced at output 216 is attenuated by an exciter attenuator circuit 218 that receives a digital command from the pulse sequence server 110. The attenuated RF excitation pulses are then applied to a power amplifier 220 that drives the RF transmission coil 204.

The MR signal produced by the subject is picked up by the RF receiver coil 208 and applied through a preamplifier 222 to the input of a receiver attenuator 224. The receiver attenuator 224 further amplifies the signal by an amount determined by a digital attenuation signal received from the pulse sequence server 110. The received signal is at or around the Larmor frequency, and this high frequency signal is down converted in a two step process by a down converter 226. The down converter 226 first mixes the MR signal with the carrier signal on line 212 and then mixes the resulting difference signal with a reference signal on line 228 that is produced by a reference frequency generator 230. The down converted MR signal is applied to the input of an analog-to-digital (“A/D”) converter 232 that samples and digitizes the analog signal. The sampled and digitized signal is then applied to a digital detector and signal processor 234 that produces 16-bit in-phase (I) values and 16-bit quadrature (Q) values corresponding to the received signal. The resulting stream of digitized I and Q values of the received signal are output to the data acquisition server 112. In addition to generating the reference signal on line 228, the reference frequency generator 230 also generates a sampling signal on line 236 that is applied to the A/D converter 232.

The basic MR systems and principles described above may be used to inform the design of other MR systems that share similar components but operate at very-different parameters. In one example, a low-field magnetic resonance imaging (IfMRI) system utilizes much of the above-described hardware, but has substantially reduced hardware requirements and a smaller hardware footprint. For example, referring to FIG. 3, a system 300 is illustrated that, instead of a 1.5 T or greater static magnetic field, utilizes a substantially smaller magnetic field. That is, in FIG. 3, as a non-limiting example, a 6.5 mT electromagnet-based scanner is illustrated. In particular, the system 300 includes a biplanar 6.5 mT electromagnet (B0) 302 that, for example, may be formed by inner B0 coils 304 and outer B0 coils 306. Biplanar gradients 308 may extend across the B0 electromagnet 302.

The system 300 may be tailored for ¹H imaging by achieving a high B0 stability, high gradient slew rates, and low overall noise. To achieve these ends, a power supply, for example, with +/−1 ppm stability over 20 min and +/−2 ppm stability over 8 h, may be used and high current shielded cables may be deployed throughout the system 300. In one non-limiting example, a power supply was adapted from a System 854T, produced by Danfysik, Taastrup, Denmark. The system 300 can operate inside a double-screened enclosure (ETS-Lindgren, St. Louis, Mo.) with a RF noise attenuation factor of 100 dB from 100 kHz to 1 GHz. In this example, the system may have a height, H, that is, as a non-limiting example, 220 cm. A cooling systems 310, such as may include air-cooling ducts, may be included.

The system 300 may be connected to a computer system to control the system 300 and reconstruct images. For example, the computer system may include the pulse sequence server 110, the data acquisition server 112, and the data processing server 114 shown in FIG. 1. The computer system may control the at least one gradient coil and the RF system to perform a MRI pulse sequence; acquire data corresponding to signals from the subject having received solution including nanodiamonds; and reconstruct, from the data, at least one anatomical image of the subject and spatially distributed nanodiamonds within the subject relative to the anatomical image.

As will be further described, the computer system may be programmed to perform at least one embedded electron paramagnetic resonance (EPR) pulse to turn on dynamic nuclear polarization (DNP) contrast caused by the solution comprising the nanodiamonds. The computer system may be programmed to deactivate the DNP contrast by turning off the at least one embedded EPR pulse. The computer system is further programmed to reactivate the DNP contrast by turning on the at least one embedded EPR pulse. The computer system may be programmed to obtain DNP data when the at least one EPR pulse is performed and reconstruct at least one DNP image from the DNP data. The computer system may further be programmed to obtain at least one difference image by taking a difference between the at least one anatomical image and the at least one DNP image.

OMRI, which is also known as proton-electron double resonance imaging, exploits the dipolar coupling between the unpaired electron of the free radical and the 1H nuclei of water to increase nuclear magnetization via dynamic nuclear polarization (DNP) and images this enhanced nuclear spin polarization with MRI. OMRI provides an excellent way to image free radical species as narrow NMR line widths enable imaging using reasonable-strength encoding gradients. OMRI also benefits from the ability to use traditional MRI sequences, though specialized hardware is needed to drive the electron spin resonance and the sequences may need to be modified to allow for EPR saturation pulses.

In OMRI, the large gyromagnetic ratio of electrons (28 GHz/T) may demand that in vivo OMRI is performed at ultra-low magnetic fields (<10 mT) in order to minimize RF heating and penetration depth issues. Operation at these magnetic fields causes a drastic reduction in NMR sensitivity despite the signal enhancement that comes from the Overhauser effect, and emphasizes the need for high S/N probes. OMRI probe design is still relatively unexplored, despite its importance, and presents challenges unique to the frequencies of operation (fH=276 kHz and fe=140.8 MHz in the OMRI experiments at 6.5 mT). High sensitivity NMR detectors and high efficiency EPR coils may be critical in decreasing the image acquisition time and boosting detection efficiency in Overhauser MRI.

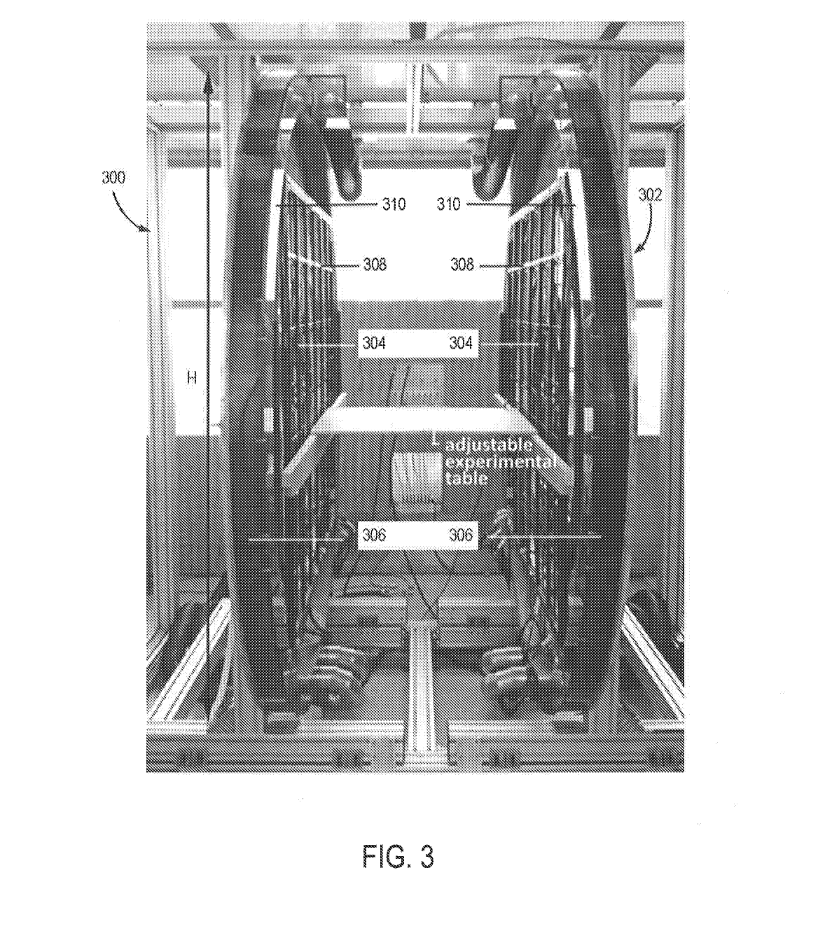

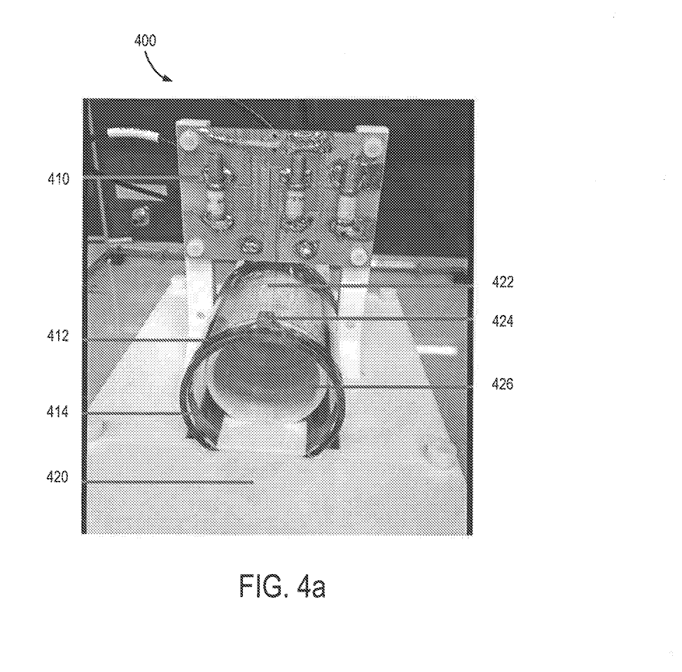

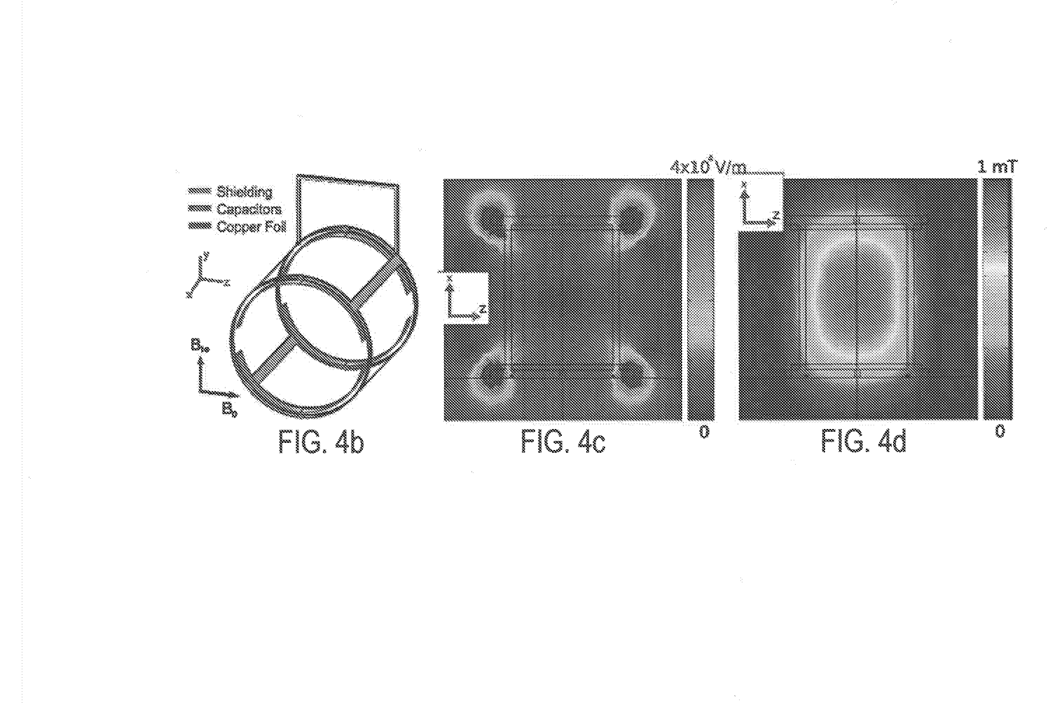

FIG. 4a shows a high performance OMRI probe 400 with broad tunability, capable of imaging enhancement over a wide range of molecular electron g-factors. The combined NMR/ESR OMRI probe 400 may be used for in vivo rat head imaging. The OMRI probe 400 may be used in combination with the system 300 in FIG. 3. The ESR resonator may be tuned to 140.8 MHz. The OMRI probe may include a litz wire NMR solenoid coil resonator board, which may be tuned to 276 kHz.

In FIG. 4a, the OMRI probe 400 includes an ESR tuning board 410 connected to frames disposed on the subject bed 420. The OMRI probe 400 further includes an NMR solenoid 422 inside a modified Alderman-Grant Resonator 412, and interfaces with the 6.5 mT electromagnet LFI (fH=276 kHz). The NMR probe design in the regime is fundamentally different to that at conventional MRI fields as thermal noise due to the intrinsic resistance of the pickup coil dominates over sample noise. This leads to a compromise where signal to noise ratio (S/N) improvements come at the expense of imaging bandwidth S/N˜Q˜1/BW.

For example, an 85 turn solenoid may be wound, using low AC resistance 5/39/42 litz wire, on a 3D printed polycarbonate solenoid former 426. This high filling factor coil has a bandwidth of 3 kHz. This modified Alderman-Grant ESR resonator 412 may be built using copper foil on Pyrex tubing. All metal placed in close proximity to the NMR solenoid 422 strongly couples, reducing the NMR sensitivity. The amount of copper used in the ESR resonator is minimized. Windows were removed from the panels on the sides of an Alderman-Grant resonator 412, a region of low current flow, to reduce coupling whilst maintaining B1 homogeneity. Shield 414 at the ends of the resonator prevents high electric fields at the capacitors 424 penetrating the imaging volume. Slits in the shielding prevent the formation of closed loops that couple to the solenoid.

FIG. 4b shows the modified Alderman-Grant resonator that includes a copper foil frame, two sets of shielding on the circular parts of the frame, and a capacitor in each shielding.

FIG. 4c shows a plot of |E| showing that the electric field is strongly suppressed inside the resonator. FIG. 4d shows the B₁S having high homogeneity in the imaging volume with less than 10% variation across the imaging region.

FIGS. 4c-d are obtained using simulations, which demonstrate the high B1 homogeneity and strong E suppression in the ESR resonator 400 shown in FIG. 4a. Testing of the new OMRI probe 400 described above indicates that it has 3× the S/N of existing probes and rectifies problems with B₁ₑ homogeneity, yielding homogeneous enhancement of −6.7 in 2 mM TEMPO when 10 W of RF power is applied.

Using the probe 400 in FIG. 4a, a new OMRI approach is provided for in vivo imaging use. The OMRI approach may employ a balanced steady state free precession (b-SSFP) sequence that embeds EPR pulses within the b-SSFP. Unlike other OMRI methods, no separate Overhauser pre-polarization step is required thus enabling fast 3D imaging. The steady-state approach also eliminates the problem of time-varying Overhauser-enhanced signal and provides constant polarization in the sample during the acquisition. The imaging may be further accelerated by Incorporating undersampled k-space strategies and compressed sensing reconstruction. This new OMRI methodology enables improved spatial and temporal resolution of free radical distribution and dynamics over current techniques.

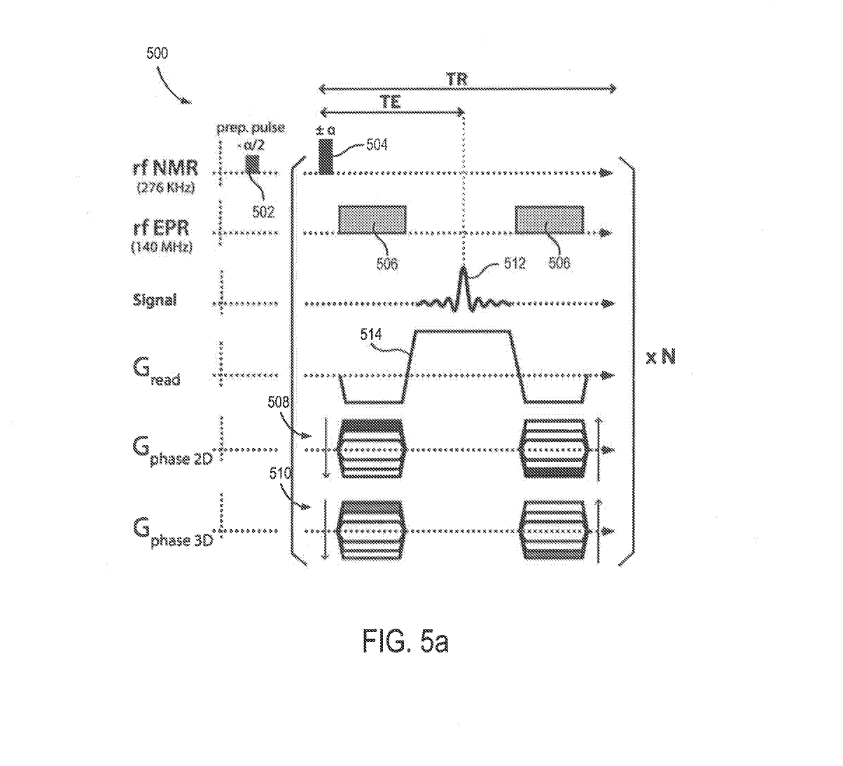

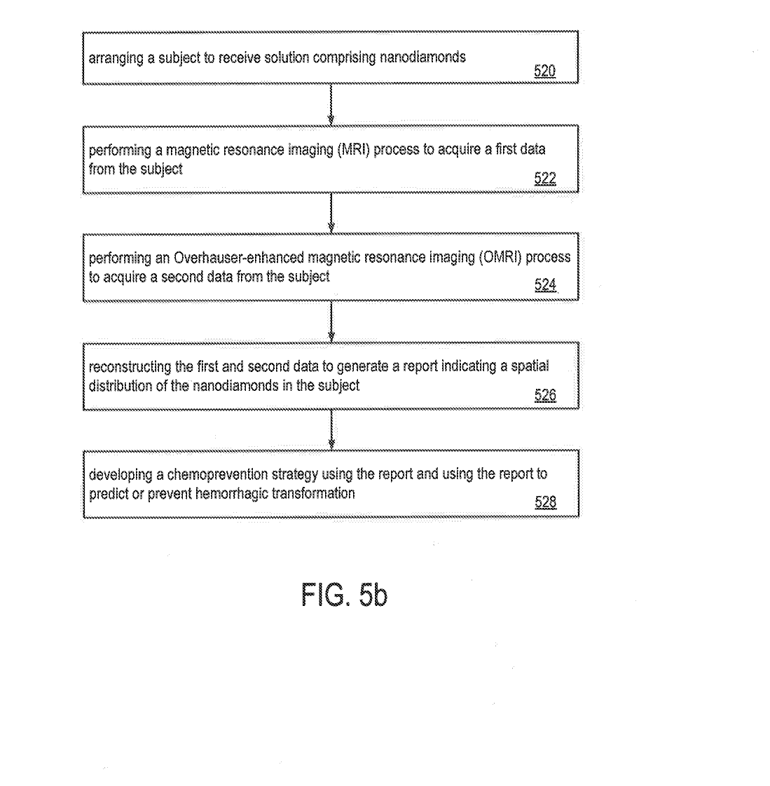

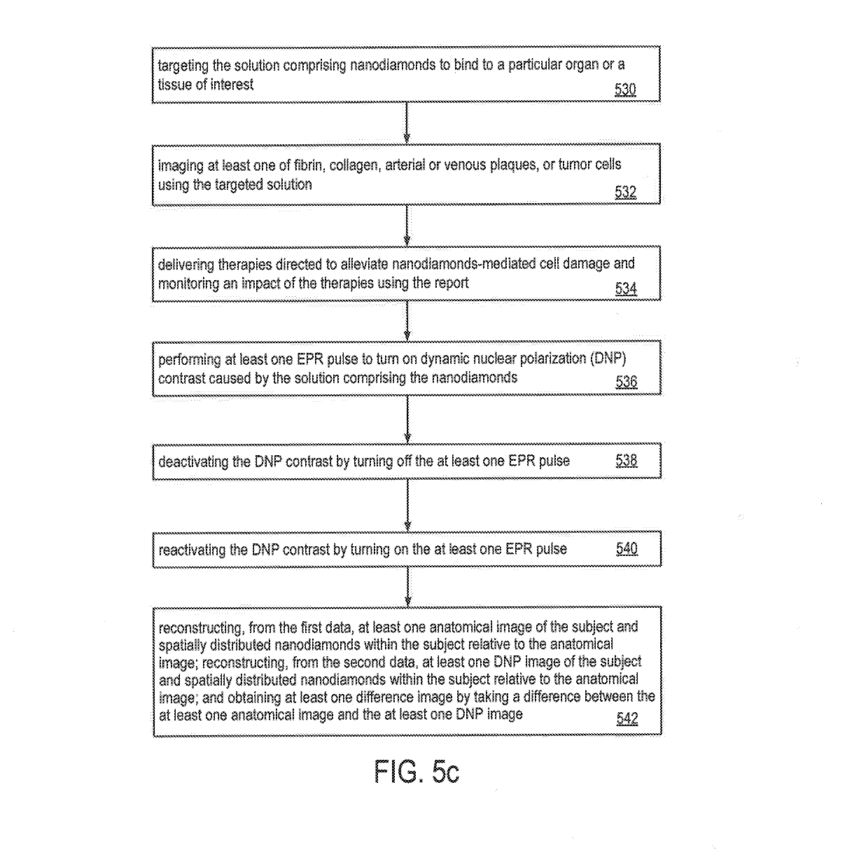

FIG. 5a is a pulse sequence diagram for a pulse sequence in accordance with the present disclosure. Referring to FIG. 5a, for imaging, a variation on a 3D balanced stead-state free procession (b-SSFP) pulse sequence 500 may be used in accordance with the present disclosure. The b-SSFP may include an initial −α/2 preparation pulse 502 followed by a train of alternating +/−α excitation pulses 504. The +1/−α excitation pulses 504 are separated by a repetition time (TR) and echo time (TE) interval between the +/−α excitation pulses 504 and the first α pulse 502 of, for example, 2 ms. One benefit of using a preparation pulse 502 is that it controls against large fluctuations of the pre-steady state signal that could produce image artifacts and thus could not be used for signal acquisition.

A selective RF excitation pulse 506 that is coordinated with a 2D phase encoding gradient pulse 508 and a 3D phase encoding gradient pulse 510 are applied to position encode the NMR signal 512 along one direction in the slice. A readout gradient pulse 514 is also applied to position encode the NMR signal 512 along a second, orthogonal direction in the slice. To maintain the steady state condition, the integrals of the gradients each sum to zero. It is important to note that, in the above-described pulse sequence 500, separate EPR saturation step is not required, unlike traditional OMRI sequences. The sequence is a b-SSFP sequence with the addition of EPR (Overhauser) irradiation 506 during the balanced phase encode gradients 508, 510, 514. Thus, no EPR saturation pulses are applied when not performing the MRI pulse sequence. Said another way, the EPR pulses are only performed during or interleaved with the MRI pulse sequence, such as the above-described b-SSFP pulse sequence.

FIG. 5b illustrates an example method in accordance with the present disclosure. The example method may be implemented using a MRI system disclosed in the disclosure. For example, in act 520, the method includes arranging a subject to receive solution comprising nanodiamonds. The subject may be a patient, an experiment subject, a phantom, etc. The subject may be arranged in a bed and may be attached to one or more receiving coils during the imaging process.

In act 522, the MRI system performs a magnetic resonance imaging (MRI) process to acquire a first data from the subject. The MRI system may perform a standard b-SSFP sequence without applying the EPR pulse.

In act 524, performing an Overhauser-enhanced magnetic resonance imaging (OMRI) process to acquire a second data from the subject. The MRI system may perform the b-SSFP sequence illustrated in FIG. 5a to acquire the second data from the subject.

In act 526, reconstructing the first and second data to generate a report indicating a spatial distribution of the nanodiamonds in the subject. The MRI system may reconstruct a first set of images using the first data and reconstruct the second set of images using the second data. The report may indicate at least one of hyper-acute or mild blood brain barrier (BBB) disruption.

In act 528, the MRI system may further develop a chemoprevention strategy using the report and use the report to predict or prevent hemorrhagic transformation. The computer system or workstation in the MRI system may implement one or more methods to analyze the report and develop treatment strategies accordingly.

FIG. 5c illustrates additional acts that may be implemented in accordance with the present disclosure. In act 530, the MRI system targets the solution including nanodiamonds to bind to a particular organ or a tissue of interest. For example, the system may identify the particular organ or the tissue of interest based on previously obtained images and target them in the MRI imaging process.

In act 532, the MRI system images the at least one of fibrin, collagen, arterial or venous plaques, or tumor cells using the targeted solution. The solution may include polymers to deliver the nanodiamonds when needed.

In act 534, the MRI system may deliver therapies directed to alleviate nanodiamonds-mediated cell damage and monitoring an impact of the therapies using the report.

In act 534, the MRI system performs at least one EPR pulse to turn on dynamic nuclear polarization (DNP) contrast caused by the solution comprising the nanodiamonds. The MRI system may deactivate the DNP contrast by turning off the at least one EPR pulse in act 536. In at 530, the MRI system may reactivate the DNP contrast by turning on the at least one EPR pulse.

In act 542, the MRI system may reconstruct, from the first data, at least one anatomical image of the subject and spatially distributed nanodiamonds within the subject relative to the anatomical image. The MRI system may reconstruct, from the second data, at least one DNP image of the subject and spatially distributed nanodiamonds within the subject relative to the anatomical image. The MRI system may then obtain at least one difference image by taking a difference between the at least one anatomical image and the at least one DNP image.

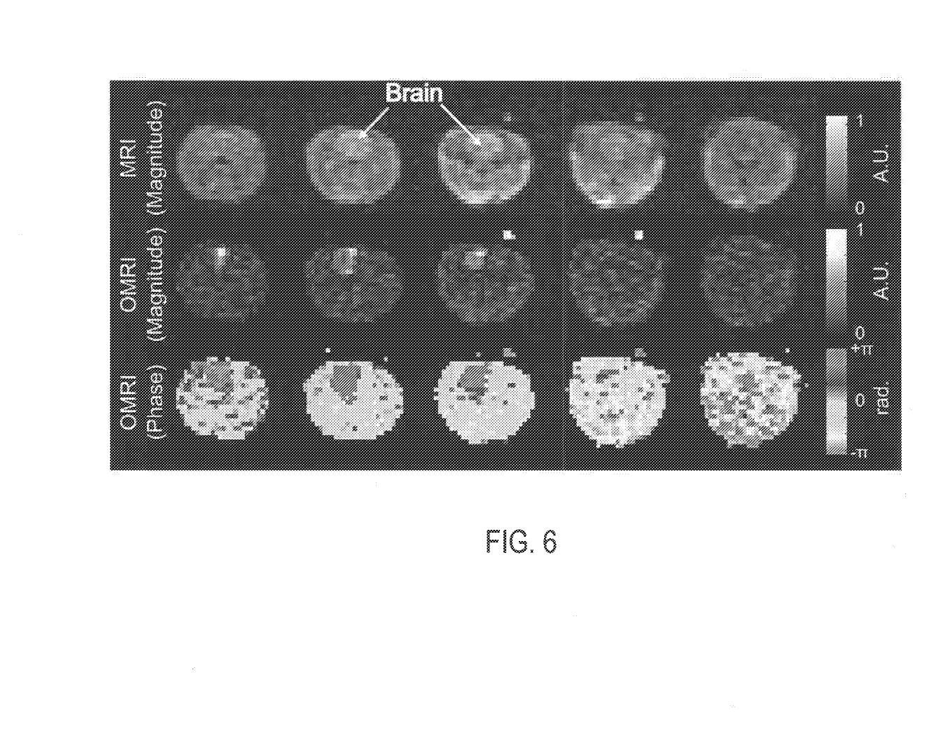

FIG. 6 shows images acquired from a rat at 6.5 mT. In the upper row, Anatomical MRI (NA=30) images are acquired in the OMRI scanner with ESR power disabled. In the middle row, OMRI images are acquired from a rat at 6.5 mT following injection of 1 mL of 150 mM TEMPOL. 5 slices of the magnitude images from an 11 slice data set are shown. The OMRI (NA=1) imaging time was 9 seconds. In the bottom row, the phase images of the 5 slices are shown.

In FIG. 6, the vivo OMRI signal enhancement is clearly visible in the rat brain after injection of the stable aqueous radical TEMPOL. As the Overhauser-enhanced signal has a phase opposite to that of the thermal signal, the phase image in FIG. 6 provides sensitive contrast in regions of low TEMPOL concentration.

The high temporal and spatial resolution in vivo shown in FIG. 6 enabled by the MRI system is a cornerstone result that allows in vivo imaging of electronic spins beyond those produced by free radicals, such as new opportunities provided by aqueous nanodiamond solutions.

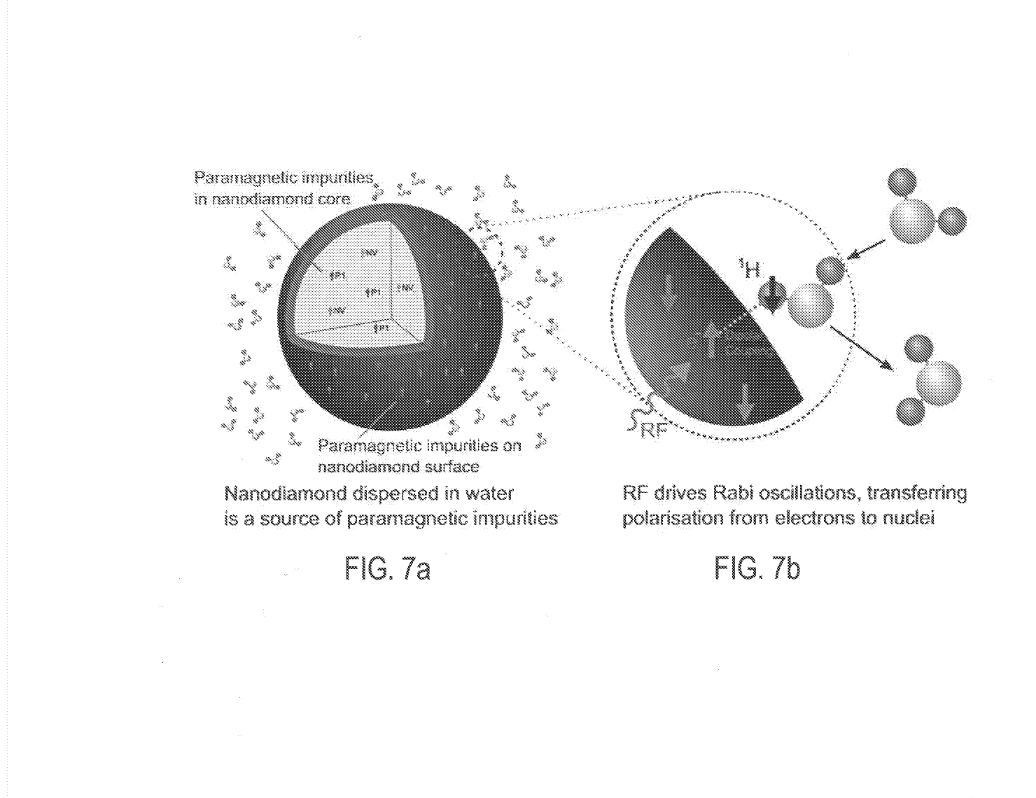

The disclosure demonstrates a DNP enhancement of the ¹H signal in water due to the presence of nanodiamonds. This enhancement is likely due to the Overhauser effect enabling polarization transfer between dangling carbon bonds at the nanodiamond surface and surrounding hydrogen nuclei. This process is illustrated schematically in FIGS. 7a-b.

FIG. 7a shows nanodiamonds dispersed in water is a source of paramagnetic impurities. FIG. 7b shows RF drives Rabi oscillations, transferring polarization from electrons to nuclei. For example, nanodiamond may contain many paramagnetic impurities such as NV centers, substitutional nitrogen (P1) centers and unpaired electrons at the surface due to dangling carbon bonds. When suspended in water, e− spins close to the ND surface interact with ¹H nuclei. The ESR transition can then be driven using RF power, transferring the high electron spin polarization to the nuclei, likely via the Overhauser effect. This increased polarization is detected using NMR and imaged with MRI in the disclosed system.

For example, the nanodiamond imaging may be performed at 6.5 mT in the ultra-low field MRI scanner in FIG. 3 at room temperature using the highly efficient b-SSFP OMRI sequence described in FIG. 5a. The system may use a custom DNP imaging probe constructed from an Alderman-Grant resonator (ESR: 191 Mhz) and a solenoid (1H: 276 kHz), similar to that shown in FIG. 4a. Spectroscopic measurements were taken in similarly constructed DNP probes with the ESR resonator tuned to fe=140 MHz for B0 sweeps or fe=191 MHz for scans at B0=6.5 mT. Synthetic NDs including MSY18 (0-30 nm NDs, median 18 nm) and MSY125 (0-250 nm NDs, median 125 nm) may be used to prepare ND/water solutions using high power probe sonication to disaggregate nanodiamond clusters. Other types of nanodiamonds may be used as well.

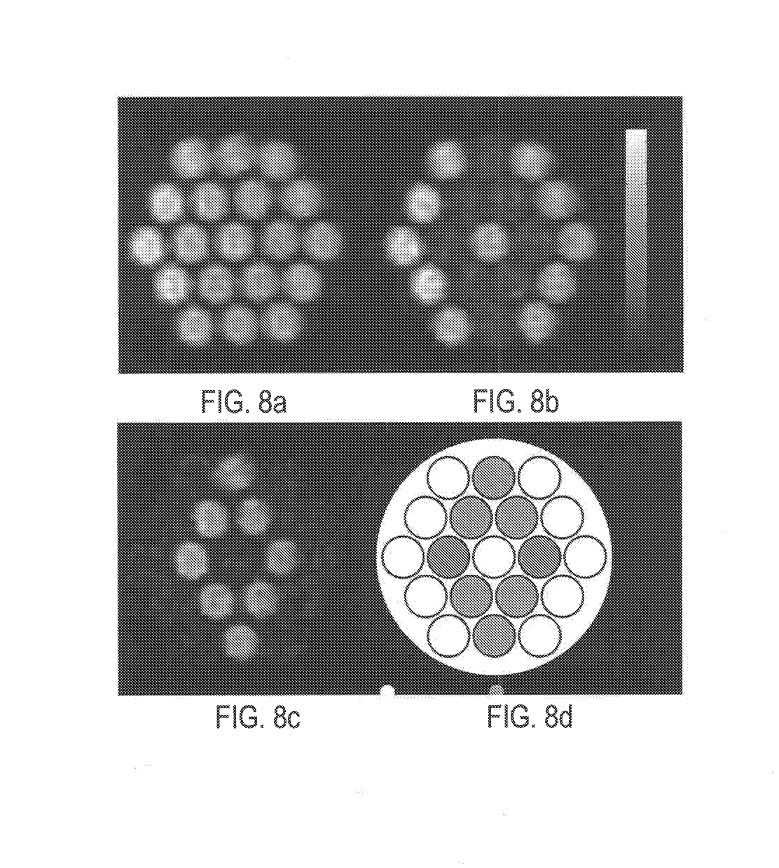

FIGS. 8a-d show MRI and OMRI images of a structured phantom, which contains an array of vials of pure water and vials of an aqueous solution of 125 nm ND solution (100 mg/mL concentration). FIG. 8a shows MRI magnitude images of a ND phantom acquired at 6.5 mT using a standard b-SSFP MRI acquisition.

FIG. 8b shows DNP MRI images of a ND phantom acquired at 6.5 mT using the OMRI b-SSFP acquisition. The 1H-e- coupling generates contrast in the MSY125 ND solution.

FIG. 8c shows the difference image of images in FIG. 8a and FIG. 8b, in which the ND solution appears bright.

FIG. 8d shows schematic of phantom vials, where white circles Indicate water-filled vials, and grey circles indicate water with ND solutions.

The Images in FIGs. 8a-d are interpolated from 0.75 mm×1 mm pixels over a 30 mm×30 mm FOV. The phantom thickness is 20 mm and the total acquisition time is 110 seconds/image. The images in FIG. 8a are acquired with a conventional b-SSFP sequence before ‘turning on’ the DNP contrast in FIG. 8b with an OMRI sequence. High contrast is demonstrated between nanodiamond solution and water, with an almost 100% reduction in the ¹H polarization with 10 W RF power applied to the ESR resonator. Similar images have been acquired for solutions of significantly smaller 18 nm NDs.

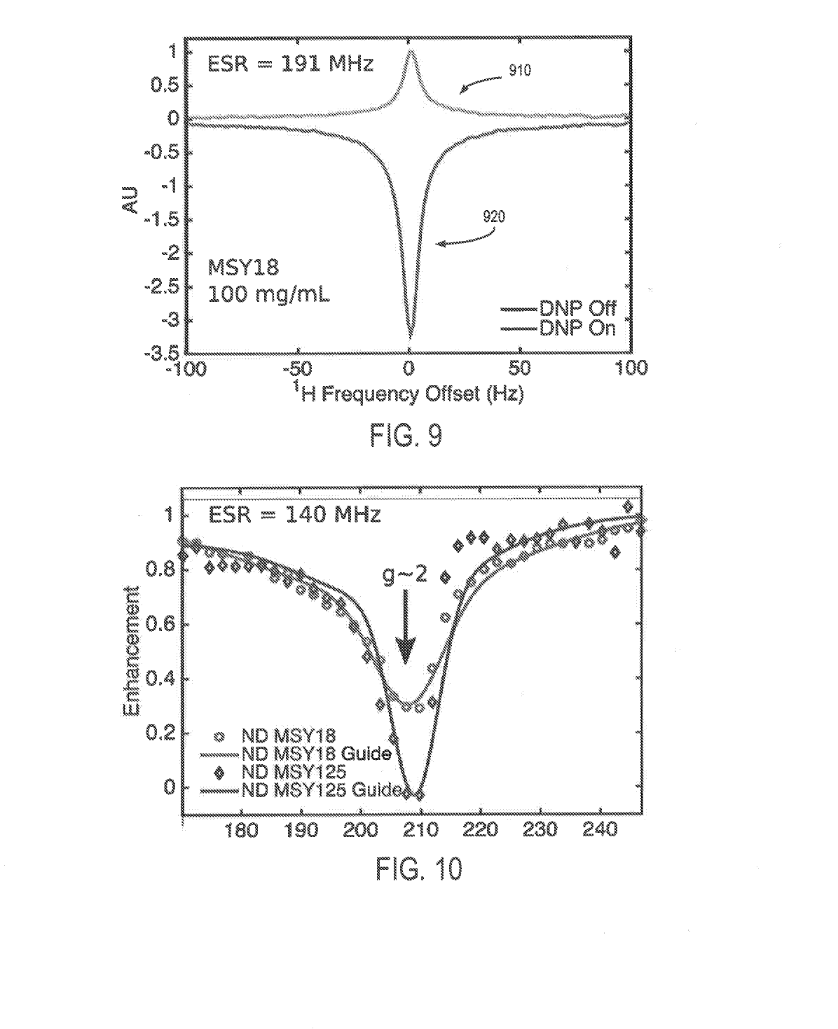

FIG. 9 shows ¹H NMR spectra at 6.5 mT from a 100 mg/mL solution of MSY18 NDs. The thermal signal in the curve 910 (green) is enhanced by a factor of −3.2 (red) in the curve 920 by driving the ESR transition at 191 MHz with 40 W of power for 500 ms.

Spectroscopic DNP measurements show that for higher RF powers, the ¹H polarization in a ND/water solution may be enhanced beyond thermal polarization. For example, an enhancement of −3.2 in the curve 920 is observed in FIG. 9. Further measurements, shown in FIG. 10, indicate the coupling between electrons and nuclei is largest when the ESR frequency corresponds to ge=−2, as expected for free electrons.

FIG. 10 shows the ¹H enhancement at a constant ESR frequency, which is measured at a series of different B0 field strengths to give an effective DNP spectrum. The solid lines in FIG. 10 are intended as a guide to the eye. ND concentration is 50 mg/mL. Based on the above results, the ND DNP may be based on surface defects. Thus, Overhauser enhancement may increase with the total nanodiamond surface exposed to water. This is consistent with the larger enhancement observed for decreasing particle size, given that the surface impurity concentration in solution is ˜1/rND, assuming spherical nanodiamonds. Further, acoustic sonication of the ND sample may break up aggregates, further increasing the amount of diamond surface exposed and improving DNP enhancement.

FIG. 10 shows a broad linewidth in the DNP spectrum, which demands a high B₁ₑ for saturation and presents a key challenge for in vivo imaging due to the SAR implications. However, this problem is somewhat mitigated by the high efficiency of the Overhauser effect at 6.5 mT (Overhauser efficiency ˜1/B0). The nontoxic, inert nature of the paramagnetic impurities also provides a clear advantage over narrow linewidth radicals conventionally used for OMRI, such as trityl or TEMPOL. Further, there is the possibility of using smaller detonation nanodiamonds (˜5 nm) and surface treatments may change water dynamics at the diamond surface [15], yielding even more sizeable polarization enhancements.

Therefore, the disclosure demonstrates the feasibility of using OMRI to image nanodiamonds in aqueous environments. The spectroscopic data indicates that the contrast in these images results from DNP via the Overhauser effect. Ultralow magnetic fields and low RF power levels minimize SAR issues and are similar to those used in free radical imaging using OMRI in vivo. The system has the ability to generate contrast across a wide range of ND sizes (18 nm vs 125 nm NDs).

The disclosed system may use NV color centers in diamond for sensitive nanoscale magnetic field sensing and imaging. The disclosed system may demonstrate diamond magnetometry. The disclosed system may obtain the first optical magnetic imaging of living biological cells the first single proton spin MRI.

The disclosure makes it possible to use the OMRI methodologies as a means of tracking ND and other nanoparticles in vivo. The enhancement attainable from nanodiamond OMRI may lead to the use of OMRI to track functionalized NDs in vivo, with a new type of DNP-based imaging contrast that can be turned “on” or “off” at will using externally applied RF pulses. Unlike optical imaging tools (such as optogenetics), which are limited by absorption and scattering to depths of about 1 mm, RF fields used in MRI are noninvasive and can fully penetrate the human body, offering fundamentally new approaches for molecular imaging. The possibility of ND OMRI may provide a new tool to investigate size-dependent cellular transport mechanisms for drug delivery.

This disclosure provides a new bio-probe based on the detection and tracking of nontoxic nanoparticles in biological environments. The system uses Overhauser-enhanced imaging methodologies to extend the diagnostic capabilities of nanodiamond to MRI. The disclosure also shows that that high contrast may be generated via the Overhauser effect due to paramagnetic impurities in the ND and enabled the demonstration the first nanodiamond-enhanced ¹H MRI in a nanodiamond/water. This result is a crucial step towards in vivo nanoparticle tracking with OMRI protocols.

The disclosure further improves the fundamental understanding of ND Dynamic Nuclear Polarization (DNP) physics, and given the already established application of ND as a biocompatible platform for drug delivery, will enable the use of MRI for nanoparticle tracking and for targeted molecular imaging and therapy.

The present disclosure has been described in terms of one or more embodiments, and it should be appreciated that many equivalents, alternatives, variations, and modifications, aside from those expressly stated, are possible and within the scope of the disclosure.

1. A magnetic resonance imaging (MRI) system, comprising:
a magnet system configured to generate a static magnetic field about at least a region of interest (ROI) of a subject arranged in the MRI system;
at least one gradient coil configured to establish at least one magnetic gradient field with respect to the static magnetic field;
a radio frequency (RF) system configured to deliver excitation pulses to the subject;
a computer system programmed to:
control the at least one gradient coil and the RF system to perform a MRI pulse sequence;
acquire data corresponding to signals from the subject having received solution comprising nanodiamonds; and
reconstruct, from the data, at least one anatomical image of the subject and spatially distributed nanodiamonds within the subject relative to the anatomical image.

2. The system of claim 1, further comprising a probe comprising a electron spin resonance (ESR) resonator tuned at a first frequency and a solenoid coil resonator tuned at a second frequency lower than the first frequency.

3. The system of claim 1, wherein the static magnetic field includes a low-field static magnetic field less than 10 mT.

4. The system of claim 1, wherein the computer system is programmed to perform at least one embedded electron paramagnetic resonance (EPR) pulse to turn on dynamic nuclear polarization (DNP) contrast caused by the solution comprising the nanodiamonds.

5. The system of claim 4, wherein the computer system is further programmed to deactivate the DNP contrast by turning off the at least one embedded EPR pulse.

6. The system of claim 5, wherein the computer system is further programmed to reactivate the DNP contrast by turning on the at least one embedded EPR pulse.

7. The system of claim 4, wherein the computer system is programmed to obtain DNP data when the at least one EPR pulse is performed and reconstruct at least one DNP image from the DNP data.

8. The system of claim 1, wherein the computer system is programmed to obtain at least one difference image by taking a difference between the at least one anatomical image and the at least one DNP image.

9. The system of claim 1, further comprising a liquid processor that prepares the solution comprising nanodiamonds using power probe sonication to disaggregate nanodiamonds clusters.

10. A method for performing a medical imaging process, comprising:
arranging a subject to receive solution comprising nanodiamonds;
performing a magnetic resonance imaging (MRI) process to acquire a first data from the subject;
performing an Overhauser-enhanced magnetic resonance imaging (OMRI) process to acquire a second data from the subject; and
reconstructing the first and second data to generate a report indicating a spatial distribution of the nanodiamonds in the subject.

11. The method of claim 9, further comprising targeting the solution comprising nanodlamonds to bind to a particular organ or a tissue of interest.

12. The method of claim 11, further comprising imaging at least one of fibrin, collagen, arterial or venous plaques, or tumor cells using the targeted solution.

13. The method of claim 10, wherein the report indicates at least one of hyper-acute or mild blood brain barrier (BBB) disruption.

14. The method of claim 10, further comprising at least one of the following:
developing a chemoprevention strategy using the report; and
using the report to predict or prevent hemorrhagic transformation.

15. The method of claim 10, further comprising delivering therapies directed to alleviate nanodiamonds-mediated cell damage and monitoring an impact of the therapies using the report.

16. The method of claim 10, further comprising performing at least one EPR pulse to turn on dynamic nuclear polarization (DNP) contrast caused by the solution comprising the nanodiamonds.

17. The method of claim 16, further comprising deactivating the DNP contrast by turning off the at least one EPR pulse.

18. The method of claim 17, further comprising reactivating the DNP contrast by turning on the at least one EPR pulse.

19. The method of claim 10, further comprising:
reconstructing, from the first data, at least one anatomical image of the subject and spatially distributed nanodlamonds within the subject relative to the anatomical image;
reconstructing, from the second data, at least one DNP image of the subject and spatially distributed nanodiamonds within the subject relative to the anatomical image; and
obtaining at least one difference image by taking a difference between the at least one anatomical image and the at least one DNP image.

20. A magnetic resonance imaging (MRI) system, comprising:
a magnet system configured to generate a static magnetic field about at least a region of interest (ROI) of a subject arranged in the MRI system;
at least one gradient coil configured to establish at least one magnetic gradient field with respect to the static magnetic field;
a radio frequency (RF) system configured to deliver excitation pulses to the subject having received solution comprising nanodiamonds, wherein the excitation pulses comprises at least one embedded electron paramagnetic resonance (EPR) pulse; and
a controller configured to manipulate dynamic nuclear polarization (DNP) contrast caused by the nanodiamonds by turning on or turning off the at least one embedded electron paramagnetic resonance (EPR) pulse.
